(19) United States
(12) Patent Application Publication
(10) Pub. No.: US 2007/0117217 A1
(43) Pub. Date: May 24, 2007

(54) Large scale parallel immuno-based allergy test and device for evanescent field excitation of fluorescence

(75) Inventors: Ratnesh Lal, Goleta, CA (US); Daniel A. Cohen, Santa Barbara, CA (US); Hai Lin, Goleta, CA (US); Arjan Quist, Lombard, IL (US); Srinivasan Ramachandran, Goleta, CA (US)

Correspondence Address: BEYER WEAVER LLP, P.O. BOX 70250, OAKLAND, CA, 94612-0250, US

(73) Assignee: The Regents of the University of California

(21) Appl. No.: 11/450,888

(22) Filed: Jun. 9, 2006

Related U.S. Application Data
(60) Provisional application No. 60/692,046, filed on Jun. 16, 2005.

(51) Int. Cl. G01N 33/563

(52) U.S. Cl. 436/513

(57) ABSTRACT

This invention provides a device and methods for the rapid detection and/or diagnosis and/or characterization of one or more allergies (e.g., causes IgE mediated allergic reaction (immediate hypersensitvity) in a mammal (e.g., a human or a non-human mammal). In certain embodiments, the device comprises a microcantilever array where different cantilevers comprising the array bear different antigens. Binding of IgE to the antigen on a cantilever causes bending of the cantilever which can be readily detected.

### CROSS-REFERENCE TO RELATED APPLICATIONS

This application claims benefit of and priority to U.S. Ser. No. 60/692,046, filed on Jun. 16, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

Not Applicable

### FIELD OF THE INVENTION

This invention pertains to the field of diagnostics. In particular, this invention provides a micro-fabricated large scale device for immunological allergy testing.

### BACKGROUND OF THE INVENTION

Three major approaches have been used in the diagnosis of allergies. These include skin tests, assays of IgE serum levels, and histamine release tests. Skin tests are the most commonly used tool for the diagnosis of allergies. The classical skin test is the Type I wheal and flare reaction assay in which antigen introduced into the skin leads to the release of preformed mediators, increased vascular permeability, local edema and itching. Such skin tests provide useful confirmatory evidence for a diagnosis of specific allergy that has been made on clinical grounds. When improperly performed, however, skin tests can lead to false positive or negative results. Particularly problematic is that a a positive reaction does not necessarily mean that the disease is allergic in nature, as some non-allergic individuals have specific IgE antibodies that produce a wheal and flare reaction to the skin test without any allergic symptoms.

The IgE-mediated false positive phenomenon observed in skin tests is not observed in in vitro methods for assaying allergen-specific IgE in patient serum (see Homburger and Katzmann (1993) Methods in Laboratory Immunology: Principles and Interpretation of Laboratory Tests for Allergy, Po. 554-572 In: Allergy Principles and Practice, Middleton et al., eds, Mosby, pub., 4th Edition, Vol. 1, Chapt. 21). Typically, allergen-specific IgE levels are measured by a radioallergosorbent test (RAST) in which a patient's serum is incubated with antigen-coated sorbent particles, followed by detection of the specific. IgE bound to antigen with labeled antibody (see, e.g., Schellenberg et al. (1975) J. Imunol., 115: 1577-1583).

Total serum IgE levels are also used in the diagnosis of allergy. Total IgE levels have typically been measured by radioimmunoassy or immunometric assay methods as described by Homburger and Katzmann, supra. IgE levels are often raised in allergic disease and grossly elevated in parasitic infestations. When assessing children or adults for the presence of atopic disease, a raised level of IgE aids the diagnosis although a normal total IgE level does not exclude atopy. The determination of total IgE alone will not predict an allergic state as there are genetic and environmental factors which play an important part in the production of clinical symptoms. The value of total serum IgE level in allergy diagnosis is also limited by the wide range of IgE serum concentrations in healthy individuals. The frequency distribution of IgE concentrations in healthy adults is markedly skewed with wide 95 percentile limits and a disproportionate number of low IgE values. Accordingly, in calculating the 95 percentile limits of normal IgE levels most investigators treat their data by logarithmic transformation, which yields upper limits for normal serum IgE that are very high when compared with arithmetic means. These high upper limits for normal serum IgE diminish the diagnostic value of the serum IgE test in screening for clinical allergy.

Histamine release tests provide a method to detect functional, allergen-specific IgE in patient serum. Typically, histamine release tests imitate the allergen-specific reaction as it occurs in the patient (see, e.g., der Zee et al. (1988) J. Allergy Clin. Immunol., 82: 270-281). This response has been generated in vitro by mixing a patient's blood with different allergens and later measuring the amount of histamine released during each of the subsequent allergic reactions. In vitro histamine release assays initially required the isolation of leukocytes from whole blood and/or various extractions of free histamine. Leukocyte histamine release tests were subsequently refined and automated to avoid cell isolation and histamine extraction (see, e.g., Siraganian et al.(1976) J. Allergy Clin. Immunol., 57: 525-540). At present, commercially available leukocyte histamine release testing kits permit up to 100 separate determinations with 2.5 ml of whole blood. However, blood samples cannot be stored for more than 24 hours prior to assay. In addition, the tests produce false positive results due to non-specific histamine release produced by toxicity of the allergen extracts or other factors. Also, a quality control study has reported considerable interlaboratory variability in the measurement of histamine (Gleich and Hull (1980) J. Allergy Clin. Immunol., 66: 295-298).

In addition, in certain patients with allergic symptoms, positive skin tests and clearly detectable IgE antibodies, no in vitro histamine release can be obtained from the patients' basophil leukocytes with allergen. This makes it impossible to interpret the results of a histamine release test if positive controls are not available and limits the usefulness of the test in diagnosing allergic disease. Levy and Osler (1967) J. Immunol., 99: 1062-1067, reported that leukocytes from only 20 to 30% of non-allergic individuals exhibit histamine release upon passive sensitization with allergen-specific IgE followed by allergen challenge in vitro. Ishizaka et al. (1973) J. Immunol., 111: 500-511expanded the usefulness of the test by showing that the incubation of leukocytes with deuterium oxide (D₂O) enhanced the histamine release induced by passive sensitization of leukocytes with anti-ragweed serum and challenge with ragweed antigen. Prahl et al. (1988) Allergy, 43: 442-448, reported the passive sensitization of isolated, IgE-deprived leukocytes from non-allergic individuals with serum from a non-releasing allergic patient followed by allergen-induced histamine release. This method, however, requires isolation of control leukocytes from the whole blood of a non-allergic donor followed by removal of IgE bound to the donor cells. Additionally, the various procedures are subject to the same histamine assay variation that limits the usefulness of the other histamine-release tests described above.

### SUMMARY OF THE INVENTION

This invention provides a device and methods for the rapid detection and/or diagnosis and/or characterization of one or more allergies (e.g., causes of IgE mediated allergic reaction (immediate hypersensitivity)) in a mammal (e.g., a human or a non-human mammal). In certain embodiments, the device comprises a microcantilever array where different cantilevers comprising the array bear different antigens. Binding of IgE to the antigen on a cantilever causes bending of the cantilever which can be readily detected.

Thus, in certain embodiments, this invention provides a device for detecting and characterizing an allergy. The device typically comprises a a sample chamber; and an array of microcantilevers where microcantilevers comprising the array have affixed thereto antigen such that there is a different species of antigen for each allergy it is desired to detect, and different species of antigen are on different microcantilevers in the array, where the free ends of the microcantilevers project into the sample chamber. In certain embodiments the device comprises at least 2, preferably at least 4, 6, or 10, more preferably at least 20, 50, 100, or 500, and most preferably at least 1,000 microcantilevers each having affixed thereto different binding moieties. In various embodiments, the device comprises one or more negative control microcantilevers treated to resist binding by protein or other moieties that can be present in a biological sample. In various embodiments the device comprises one or more positive control microcantilevers having attached thereto an antibody that binds IgE antibodies. In certain embodiments the antibody that binds to IgE antibodies is a single chain antibody, or a full antibody, or an antibody fragment. In certain embodiments the antibody can be a monoclonal or a polyclonal antibody. The device can optionally further comprise a first means of detecting deflection of a cantilever when binding moieties on the cantilever bind a target analyte and it can optionally comprise a second means of detecting deflection of a cantilever when binding moieties on the cantilever bind a target analyte. In various embodiments the first means and the second means, when present, are independently selected from the group consisting of a piezoresistive detection means, a piezoelectric detection means, and an optical detection means, the latter of which comprises means to detect optical beam deflection, optical phase shift, optical intensity shift, and/or evanescent field excitation of fluorescence. In certain embodiments the allergen is selected from the group consisting of a pet allergen, dust, mold spores, pollen, a food allergen, and an insect bite allergen.

In various embodiments this invention provides a method of identifying an allergy in a subject. The method typically involves providing a biological sample from the subject comprising IgE antibodies; and contacting the biological sample or a component thereof with a microcantilever device as described herein; and detecting deflection of one or more cantilevers in the microcantilever array in response to binding by IgE where binding of the cantilever indicates that the subject has an allergic response to the antigen present on the deflected cantilever. In certain embodiments the detecting comprises a method selected from the group consisting of detecting an optical signal, detecting a piezoresistive signal, detecting an optical signal, detecting an evanescent wave signal. In certain embodiments the detecting comprises utilizing at least two different detection methods. In various embodiments the sample comprises whole blood, plasma, serum, lymph, oral fluid, or cerebrospinal fluid.

In still another embodiment, this invention provides a device for detecting the presence, absence, or quantity of a plurality of analytes. The device typically comprises a sample area or chamber; and an array of microcantilevers where micocantilevers comprising the array have affixed thereto binding moieties such that there is a different species of binding moiety that specifically or preferentially binds each species of analyte that is to be detected; and different species of binding moiety are on different microcantilevers in the array, where the free ends of the microcantilevers project into the sample chamber. In certain embodiments the device comprises at least 2, preferably at least 4, 6, or 10, more preferably at least 20, 50, 100, or 500, and most preferably at least 1,000 microcantilevers each having affixed thereto different binding moieties. Suitable binding moieties include, but are not limited to a nucleic acid, an antibody, a receptor, a carbohydrate, a protein, a glycoprotein, and the like. The device can optionally further comprise a first means of detecting deflection of a cantilever when binding moieties on the cantilever bind a target analyte and it can optionally comprise a second means of detecting deflection of a cantilever when binding moieties on the cantilever bind a target analyte. In various embodiments the first means and the second means, when present, are independently selected from the group consisting of an optical detection means, a piezoresistive detection means, a piezoelectric detection means, and an evanescent wave detection means.

This invention also provides improved devices for use in total internal reflectance microscopy (TIRFM). Thus, in certain embodiments, this invention provides a device for supporting a sample and for providing evanescent field excitation of fluorescence in total internal reflectance microscopy (TIRFM), the device comprising: a substantially planar optical waveguide comprising two substantially parallel surfaces; and an active optical coupler affixed or juxtaposed to the waveguide such that light generated from the coupler enters the waveguide, where the active optical coupler is not a fluorophore. In certain embodiments the device is a device for supporting a sample and for providing evanescent field excitation of fluorescence in total internal reflectance microscopy (TIRFM), the device comprising: a substantially planar optical waveguide comprising two substantially parallel surfaces; an active optical coupler affixed or juxtaposed to the waveguide such that light generated from the coupler enters the waveguide; and an angle filter comprising a material whose refractive index is between that of the waveguide and air, where the angle filter is disposed on a surface of the waveguide to substantially reduce light propagating in the waveguide. In certain embodiments the device is a device for supporting a sample and for providing evanescent field excitation of fluorescence in total internal reflectance microscopy (TIRFM), the device comprising a substantially planar optical waveguide comprising two substantially parallel surfaces; and a passive optical coupler affixed or juxtaposed to the waveguide such that light provided from the coupler enters the waveguide. In certain embodiments the active optical coupler is an electrically driven coupler or an optically pumped laser. In various embodiments the active optical coupler is an electrically driven coupler selected from the group consisting of a light emitting diode (LED), and a laser diode. In various embodiments the active optical coupler is a fluorophore. In certain embodiments the passive optical coupler is selected from the group consisting of a lens, a prism, a facet, a grating, a mirror, a gradient index structure, and a scattering structure. In various embodiments the device further comprises an angle filter comprising a material whose refractive index is between that of the waveguide and air, where the angle filter is disposed on a surface of the waveguide to substantially reduce light propagating in the waveguide. : In various embodiments the angle filter substantially eliminates or reduces light propagating in the waveguide at an angle below some critical angle, measured relative to a line perpendicular to the waveguide surface and drawn into the waveguide, said angle ranging from about 35 degrees to about 70 degrees, depending on use. In various embodiments the waveguide has an index of refraction of about 1.4 or more. In certain embodiments the waveguide ranges in thickness from about 50 μm to about 1 mm, preferably from about 50 μm to about 500 μm, more preferably from about 100 μm to about 200 μm. Suitable waveguides typically comprise a material selected from the group consisting of glass, plastic, and a crystalline material (e.g., quartz, sapphire, silicon carbide, calcium fluoride, aluminum nitride, gallium nitride, aluminum gallium nitride, lithium niobate, etc.). In certain embodiments the waveguide comprises a coverslip.

In certain embodiments the optical coupler is laminated, chemisorbed, or cemented to the waveguide. In certain embodiments the optical coupler is fabricated in situ on the waveguide. In various embodiments the devices optionally further comprise a means (e.g., a reservoir, a pedestal, a well, etc.) for supporting or affixing a sample such that all or a portion of the sample is exposed to an evanescent field from the optical waveguide. The devices can optionally further comprise a means to measure intensity of an excitation light (e.g., an evanescent field). In certain embodiments the means to measure excitation intensity comprises one or more fluorophores that are excited by the same evanescent field used to excite the sample of interest, and that emit fluorescence that is proportional to excitation intensity. The fluorophores can be distributed on the waveguide surface in known and easily distinguishable patterns or in random and/or haphazard patterns. In certain embodiments the means to measure excitation intensity comprises a photodiode that intercepts a portion of the excitation light (e.g., evanescent field). The devices can optionally further comprise a means to quantify sample distance from the waveguide surface. In certain embodiments the means to quantify sample distance comprises fluorescent markers at known distances from the waveguide surface. In certain embodiments the means to quantify sample distance comprises two or more couplers emitting light at significantly different wavelengths, in conjunction with a sample fluorophore that can be excited by light at significantly different wavelengths. The devices can also include structures that reduce scattering of excitation light at boundaries of fluids disposed on the waveguide surface, or at boundaries of structures that contain those fluids. In certain embodiments the structures comprise an antireflection layer and/or an absorption layer. In certain embodiments the structures are selected from the group consisting of structures fabricated from material with an index of refraction approximately equal to that of the contained fluid, and structures with reentrant profiles such that light scattered at the point of contact between the structure and the substrate is subsequently intercepted and absorbed by another part of the structure. In various embodiments the planar surface opposite the sample is coated with a smooth and transparent layer of thickness greater than approximately one micrometer and index of refraction lower than that of the waveguide, such that light trapped by total internal reflection in the waveguide does not penetrate evanescently to the surface of the layer. Thus, in various embodiments the device further comprises a substantially planar low refractive index material immediately below the waveguide. Typically the low refractive index material has a refractive at least 0.02, preferably at least 0.05, and more preferably at least 0.10 below that of the waveguide, and a thickness of at least 1 μm, preferably at least 2 μm, more preferably at least 5 μm or 10 μm. In certain embodiments a solid or liquid layer is disposed on the substrate such that excitation light propagating within the waveguide within some range of propagation angles relative to the planar surface is transmitted out of the substrate and into the solid or liquid layer, and is subsequently transmitted away from the device or absorbed. In various embodiments a planar surface opposite the sample is coated with an absorptive or reflective optical filter, such that only sample fluorescence of selected wavelengths is transmitted through the filter.

In certain embodiments the active coupler is not a fluorophore. The exclusion is not intended to exclude the use of a fluorophore inside an optically pumped laser, where it emits by stimulated emission, not spontaneous emission. Thus, unless otherwise specified the exclusion only eliminates fluorophores where they emit by spontaneous emission. In certain other embodiments fluorophores that emit by stimulated emission are excluded.

Definitions

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H-}V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No: 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The term “preferentially binds” refers to a moiety that binds to a particular target with greater affinity or avidity than to other targets present in the same sample. Preferential binding thus provides a means by which the presence and/or quantity of the target analyte (e.g., a particular IgE) is present in a sample.

The term “sample” or “biological sample” when used herein in reference, e.g. to an allergy assay refers to a sample of a biological material that typically contains IgE antibodies. Such samples include, for example, whole blood, serum, etc. The sample can be a “raw” sample simply as taken from a subject or the sample can be processed, e.g. to remove cellular debris.

The term “allergy” refers to a condition in which the body has an exaggerated response to a substance (e.g., mold spores, pollen, insect toxins, animal dander, certain drugs and food, etc.). Also known as hypersensitivity.

An “allergen” refers to a substance that induces an allergic response.

The term “antigen” refers to a substance, typically foreign to the body, that stimulates the production of antibodies by the immune system. Antigens include foreign proteins, bacteria, viruses, pollen, and other materials.

An optical coupler refers to a device that can introduce light into a waveguide. Optical couplers include, but are not limited to active optical couplers that generate a light in response to, e.g. an electrical or optical input, and passive optical couplers that simply scatter, reflect, or otherwise redirect an incident light.

High angle light refers to light that would emerge from the optical waveguide planar surfaces.

### BRIEF DESCRIPTION OF THE DRAWINGS

FIG. 1 illustrates one embodiment of a cantilever array of this invention. Dimension d is typically about 1 to 3 cm.

FIG. 2 illustrates individual cantilevers comprising an array.

FIG. 3 illustrates cantilevers in a fluid cell.

FIG. 4 illustrates binding of IgE to antigens crosslinked on the cantilever surface induces displacement (bending) of the cantilever.

FIG. 5 provides one illustrative configuration of a microcantilever array showing “test” cantilevers as well as positive and negative control cantilevers for each of three antigens the array is designed to detect.

FIGS. 6A and 6B illustrates various possible configurations of microcantilever arrays of this invention. FIG. 6A illustrates dual cantilevers projecting into two sample chambers. FIG. 6B illustrates a multi-planar configuration of a microcantilever array.

FIG. 7 illustrates a detection system based on reflection of a light source (e.g., a laser) off of the microcantilever(s).

FIG. 8 schematically illustrates one embodiment of a device for evanescent field excitation of fluorescence according to methods

FIG. 9 schematically illustrates one embodiment of a device for evanescent field excitation of fluorescence according to methods described herein.

FIG. 10 panels A, B, and C, illustrate different geometries for LED and waveguide structures. LED electrical contacts are shown in light and dark grey.

FIG. 11 schematically illustrates one embodiment of an evanescent field based detection system as described herein in use on an inverted light microscope.

### DETAILED DESCRIPTION

In certain embodiments this invention provides a device and methods for the rapid detection and/or diagnosis and/or characterization of one or more allergies (e.g., causes of IgE mediated allergic reaction (immediate hypersensitvity)) in a mammal (e.g., a human or a non-human mammal). Instead of testing one or a few allergens at a time as in traditional methods, this devices and methods described herein allow simultaneous examinations of hundreds of allergens. The methods are fast, economical, and significantly reduce the discomforts of patients. Typically the assay takes only a few minutes and requires less than 1 ml of blood sample.

Currently, there are two predominant allergy tests for immediate hypersensitivity: the skin test and a test for allergy specific IgE in blood serum. During skin tests, potential allergens are placed on the skin and the reaction is observed. To detect allergen-specific IgE in serum, a patient's blood serum is combined with allergen attached to a substrate in a test tube, and radioactive-labeled (Radioallergosorbent test, RAST) anti-IgE antibody is added later to determine if serum IgE reacts with the allergen (the secondary antibody can also be labeled with chemiluminescent/ fluorescent markers).

Though the skin test is generally considered to be the most reliable test for allergens, due to the large numbers of different allergens in our environment, it is time consuming, expensive, and impractical to perform skin tests for a small amount of many different allergens present. A large number of skin tests to test a wide variety of allergens also can cause significant discomfort in the patients and in rare cases, the skin tests can induce anaphylaxis, a sever allergic reaction that can be life threatening.

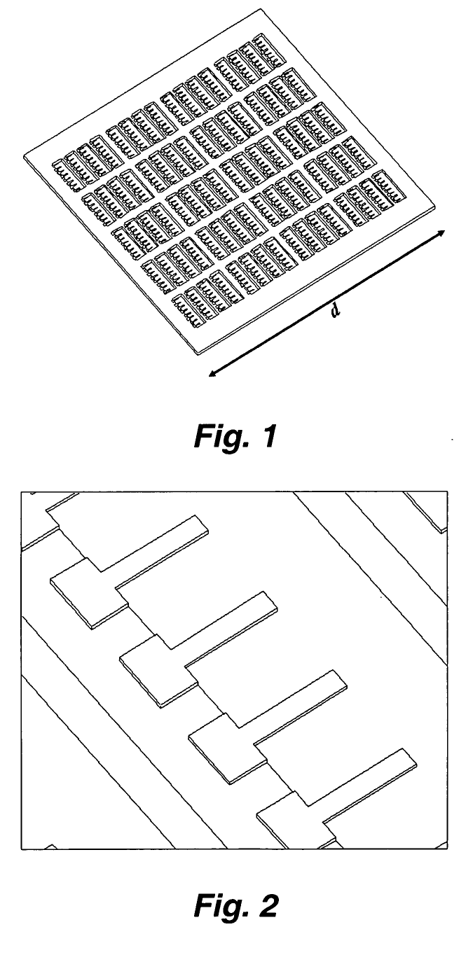

In various embodiments the allergen assays of the present invention utilize a micro-fabricated cantilever array (see, e.g., FIGS. 1 and 2) and a detection system to monitor the displacement of the free end(s) of the cantilevers. The micro-cantilevers can readily be fabricated using various solid state fabrication techniques (e.g., photolithography). In various embodiments, each cantilever comprises is a slender beam with one end attached to a support base (see, e.g., FIG. 2). Various cantilevers comprising the microcantilever array have affixed thereto antigen(s) such that each cantilever that bears antigen bears a single species of antigen. Different cantilevers comprising the array can bear different species of antigen and the entire array typically comprises a plurality of different antigen species. Thus, in certain embodiments the cantilever array comprises at least two, preferably at least 5 or 10, more preferably at least 20 or at least 50, still more preferably at least 75 or at least 100, and most preferably at least 150, 200, 500, or at least 1000 different antigen(s).

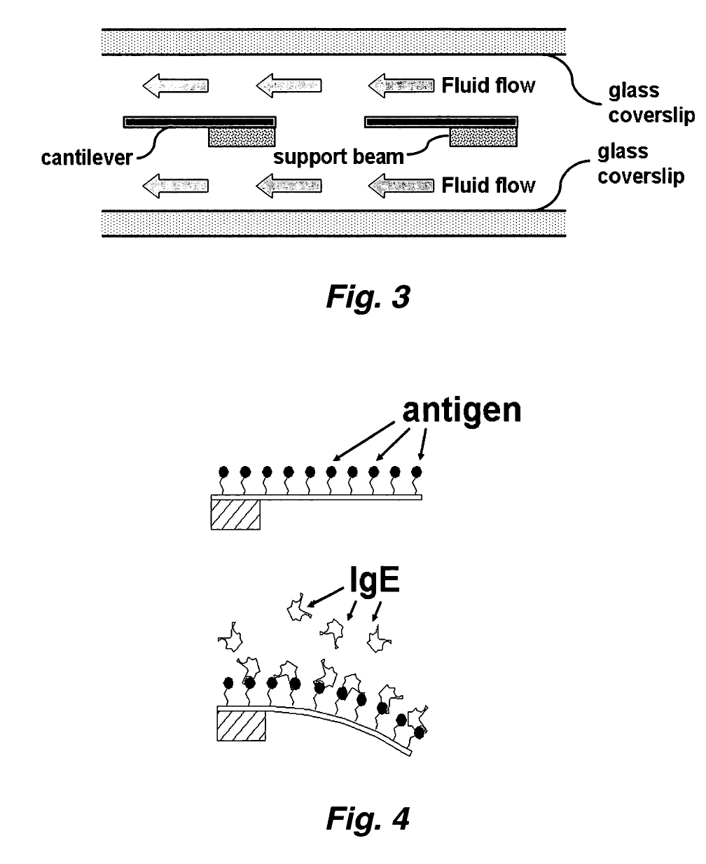

In certain embodiments the cantilever array is placed in a small fluid cell (e.g., volume<0.2 cm³), that allows perfusion and exchange of fluid (see, e.g., FIG. 3). In certain embodiments the fluid cell is bound by optically transparent surfaces (e.g., thin glass coverslips) on top and bottom, that allow optical access to the cantilevers. The temperature of the fluid perfusion system and fluid cell typically can be controlled and monitored. An optional fluorescence microscopic imaging system, e.g., as described herein, can be attached.

On each “test” cantilever, species of a specific purified allergen extract is attached to, e.g., covalently linked to the cantilever. In certain embodiments the allergen is just linked to one side of the micro-cantilever, while the opposite side is chemically modified to prevent the attachment of any protein.

If a subject is allergic to a specific allergen, then an IgE should be present in his/her blood serum that binds specifically to that allergen. When the blood serum is introduced, specific IgEs will bind to their respective target allergens on the each of the many cantilevers in the cantilever array. The preferential binding at specific cantilevers will induce a displacement (bending) at the free ends of these cantilevers (see, e.g., FIG. 4). The displacement of a certain cantilever indicates the presence of an IgE subclass specific for the attached allergen. The displacements of the cantilevers can be detected and optionally quantified, by any of a variety of methods, e.g. using optical positioning/detecting methods, by fabricating the cantilevers with piezo-resistant or piezoelectric materials, and the like.

In various embodiments an array of cantilevers can include certain cantilevers assigned as “test” cantilevers and certain cantilevers assigned as “control” cantilevers. The “test” cantilevers typically carry the antigen used to detect the IgE binding.

The “control” cantilevers can include positive control and/or negative control cantilevers. Surfaces of the negative control cantilevers are typically chemically modified so that proteins do not attach to these cantilevers. Using negative control cantilevers mounted nearby as references, it is possible to subtract and thus minimize the cantilever movement due to fluid disturbance and temperature fluctuations.

Positive control cantilevers typically have anti-IgE antibody attached to the cantilever. The anti-IgE will bind to all IgE molecules. Using these positive controls, the sensitivity of the detection system can be calibrated.

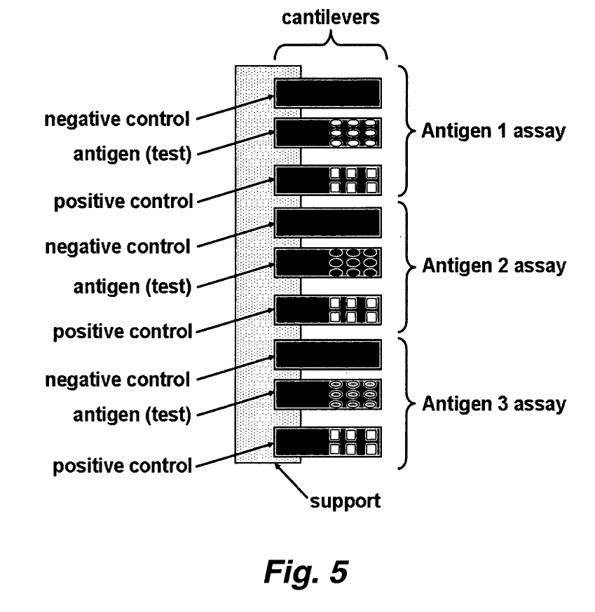

Essentially any cantilevers comprising the microcantilever array can be utilized for “test” or positive and/or negative “controls”. In certain embodiments there may be only a few positive and/or negative control cantilevers and a large number of “test” cantilevers, i.e., the test cantilevers may outnumber the control cantilevers, e.g., by a factor of at least 1.5, 2, 3, or even 4, or 5 or more. In certain embodiments each “test” cantilever has an associated positive and/or negative control cantilever. Thus, for example, as illustrated in FIG. 5, each “test” cantilever can have an associated positive and negative control cantilever.

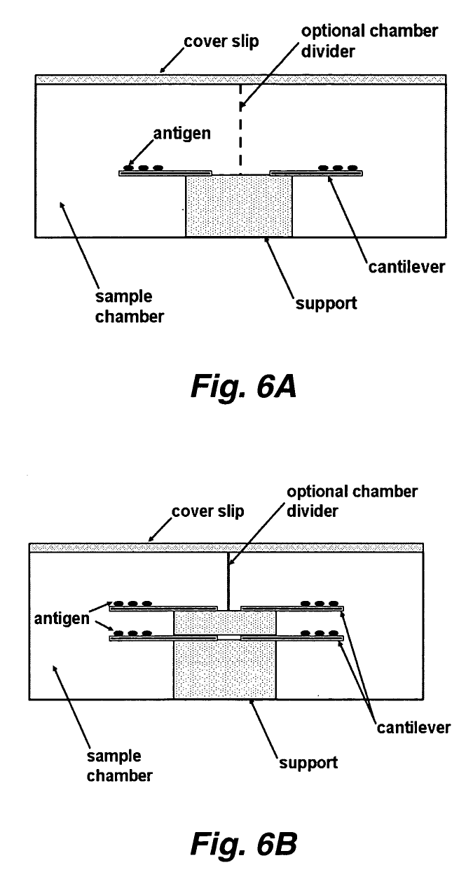

While the microcantilever arrays of this invention are generally illustrated as single planar arrays, it will be appreciated that other geometries are also suitable. Thus, for example, FIG. 6A illustrates microcantilevers projecting off of both sides of a support. The support can, optionally be bisected with an optional barrier to form two separate sample chamber. In certain embodiments the microcantilevers need not be limited to a single plane. Thus, for example, 6B illustrates microcantilever arrays comprising cantilevers in two planes.

The foregoing embodiments, are intended to be illustrative and not limiting. Using the teaching provided herein, other suitable embodiments will be apparent to one of skill in the art.

Detection.

Bending of the cantilever(s) can be detected and, optionally quantified, by any of a number of methods known to those of skill in the art. Thus, for example in one simple embodiment, bending can be determined by simple visualization of beam deflection (e.g. using a microscope). In certain embodiments beam deflection can be further analyzed using optical microscopy accompanied by digital image analysis.

In certain embodiments cantilever deflection can be measured by a change in conductivity of a metallic or semiconducting strain gauge that is formed on the top and/or bottom surface of the microcantilever(s) during the microfabrication process. In addition or alternatively, the cantilever(s) can be fabricated out of a piezo electric or a piezo resistive material and bending can be measured by the creation of a potential and/or a change in resistance of the device.

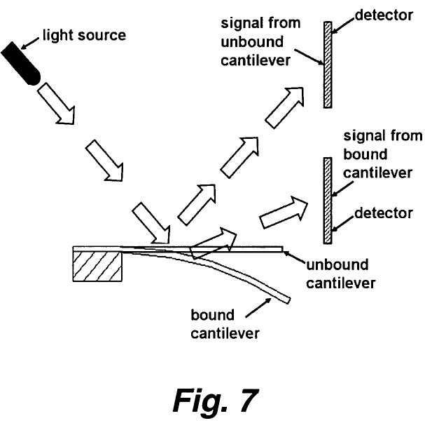

Beam deflection can also be measured by various reflective and/or interferometric methods. Thus, for example, in one embodiment illustrated in FIG. 7a light source (e.g., a laser) can be directed at the cantilever(s) and the reflected beam detected, e.g. using a photomultiplier, a CCD device, or other detector. When the beam bends, the reflected light will move or change intensity thereby providing a measure of beam detection.

In certain embodiments the detection of allergen specific IgE can be further enhanced by using a secondary antibody. After the binding of specific IgE to cantilevers, the cantilever array is washed with a saline buffer to remove unbound IgE molecules, followed by the perfusion of anti-IgE antibody into the fluid cell. Anti-IgE antibody will bind preferentially to cantilevers with IgE attached, which will also induce displacements on selective cantilevers and indicate which of the allergen-specific IgE is present. If fluorescently conjugated anti-IgE antibody is used, the binding of anti-IgE antibody to IgE can also be confirmed

In certain embodiments, detection can involve evanescent field excitation as described herein.

Improved Devices for Total Internal Reflectance Microscopy.

In various embodiments, microscopic methods can be used to detect and/or quantify displacement of the microcantilever(s). In certain preferred embodiments the microscopic methods include, but are not limited to total internal reflection fluorescence microscopy (TIRFM). In TIRFM light trapped within a waveguide by total internal reflection produces an evanescent optical field at the surface of a waveguide. The evanescent optical field at the surface of the waveguide is used to illuminate, e.g., excite fluorescence in molecules, particles, objects (e.g., microcantilevers), or cells of interest that are in close proximity to the waveguide surface, without exciting fluorescence in species further away from the waveguide surface.

While TIRFM methods are well known to those of skill in the art, in various embodiments this invention provides improved TIRFM methods. In particular, in certain embodiments, this invention provides improved device for evanescent field illumination and/or excitation of fluorescence. The present invention eliminates both the expense and alignment issues associated with traditional TIRFM methods, allowing TIRFM measurements using simple optical microscopes.

In microscopy applications, it is particularly important to limit the amount of excitation light that is scattered out of the waveguide and into the region containing the sample of interest, such as into serum, cell growth media, buffer fluids used for sample transport, microdevices, and the like. Such scattered light may excite fluorescence in other sample components, similar or dissimilar to that component under observation. The additional fluorescence will degrade the signal-to-background ratio and limit the ability to observe or measure features in the sample of interest. Certain innovations of this invention are structures to limit this scatter.

In evanescent field microscopy or sensing, the distance the evanescent optical field penetrates into the sample depends on the effective angle of incidence of the excitation light onto the surface used for total internal reflection, such as a coupling prism. In the current art, this angle of incidence is determined by adjustment of optical components and must be carefully calibrated in use. Another innovation of this invention is to use a simple filter, fixed during manufacture or variable by the user, to select from a range of angles of incidence, resulting in evanescent field penetration depths that may be reliably controlled.

For quantitative TIRF microscopy or sensing, or to monitor sample fluorescence over time, the intensity of the excitation light must is preferably controlled or monitored. An innovation of this invention is to include means integrated onto the waveguide surface to monitor the excitation intensity. In certain implementations, the monitor comprises fluorophores that are efficiently excited by the same light used to excite the sample fluorophores of interest, and which emit fluorescence that is proportional to excitation intensity over a wide range of excitation intensities. In various embodiments the fluorophores are selected that are stable with respect to time, temperature, total exposure, and chemical environment. The monitor fluorophores can optionally be covered in a passivating layer to improve any of these properties, in which case, the index of refraction of the passivating layer should be close to that of the sample or a fluid containing the sample. The monitor fluorophores may emit at wavelengths overlapping the sample fluorescence, in which case the monitor fluorophores should be distributed on the waveguide surface in known and easily distinguishable patterns. Alternatively, the monitor fluorophores may emit at wavelengths easily separated from both the sample fluorescence and the excitation wavelengths by spectral filtering, in which case the monitor fluorophores may be distributed randomly over the waveguide surface. An alternative means to monitor the excitation intensity is to incorporate a monitor photodiode or other optical detector onto the waveguide surface, such that a signal (e.g., an electrical signal) proportional to the excitation intensity is generated.

In TIRF microscopy, it is often desirable to measure the distance a sample fluorophore (e.g., a biological cell or cell component, a microcantilever, a microcantilever bearing a fluorophore, etc.) is from the surface, and monitor this distance over time or in response to stimulus. When a sample contains a stable fluorophore, the fluorescence intensity serves as a relative indication of distance from the surface of total internal reflection, in this case, the waveguide surface. An innovation of this invention is to incorporate fluorescent markers identical or similar to the fluorescent species in the sample, at known distances from the waveguide surface, to serve as distance markers for quantitative distance measurement. Thin layers of such fluorophores can be disposed directly on the waveguide surface, and/or on transparent films of known thickness above the waveguide surface, in regions small compared to the microscope field of view, so that one or more such distance calibration markers are always visible.

An alternative means for quantitative measurement of the distance from the waveguide surface relies on the wavelength dependence of the evanescent field penetration depth: longer wavelengths penetrate further. Thus, a fluorophore that can be excited with significantly different wavelengths will fluoresce relatively brighter with longer wavelength excitation, after appropriate calibration for absorption coefficient and quantum yield.

In certain embodiments two or more fluorophores having different excitation wavelengths are or fluorophores having two or more different excitation wavelengths are incorporated. In such embodiments the ratio of fluorescence intensity from different excitation wavelengths can be used to calculate a sample fluorophore's distance from the waveguide surface.

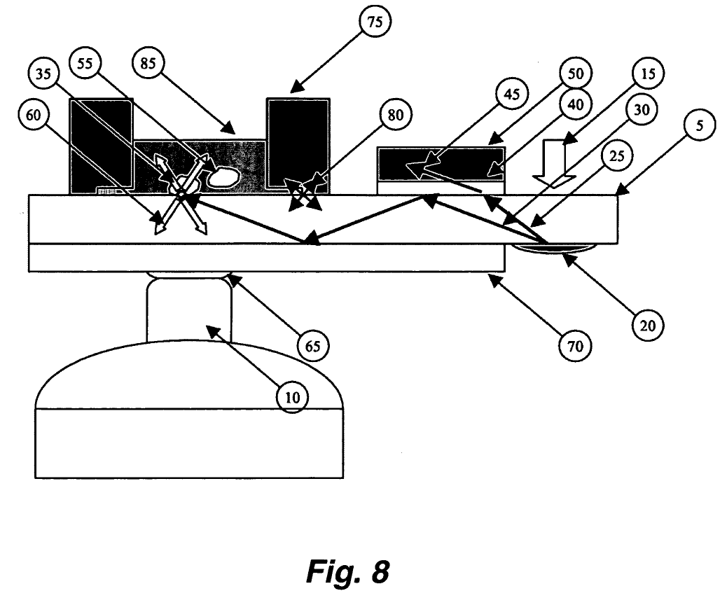

One illustrative implementation of the invention is shown schematically in FIG. 8. A substantially planar optical waveguide 5, comprising two smooth and approximately parallel surfaces, is fabricated from a material transparent to both excitation and emission wavelengths of the sample fluorescence to be observed. Typically the optical waveguide has a refractive index greater than about 1.2 or 1.4, preferably greater than about 1.6, and more preferably than about 2.0, with a likely range between about 1.4 and 2.4).

The thickness of the planar waveguide typically ranges from about 25 μm to about 1 mm, preferably from about 50 μm to about 400 μm or 300 μm, more preferably from about 75 μm to about 250 μm, and most preferably from about 100 μm to about 200 μm. In certain embodiments the thickness of the planar waveguide is approximately 100-200 micrometers, suitable for high magnification viewing with common inverted microscope objective lenses 10.

A primary excitation source, in this implementation light of wavelength 1 15, is directed onto an active coupler, in this implementation a pump fluorophore 20 that is efficiently excited at wavelength 1 and efficiently emits at wavelength 2. Light emitted from this fluorescent optical coupler is coupled into the planar substrate at a multitude of angles. Some of this light exits from the waveguide, and some is trapped by total internal reflection within the waveguide, shown here as two rays 25 and 30. Disposed upon at least one planar surface of the waveguide and positioned between the light coupler 20 and the sample to be viewed 35 is a material 40 whose refractive index is between that of the waveguide and the medium containing the sample, (typically 1,0 when the sample is contained in air or 1.34 when the sample is contained in water), chosen to achieve a particular critical angle for total internal reflection (e.g., typically from about 35 degrees to 70 degrees, measured relative to a line perpendicular to the waveguide surface and drawn into the waveguide). Light incident upon the interface between the waveguide and the material 40 at angles below the critical angle will be partially transmitted out of the waveguide into material 40. Light incident at angles greater than the critical angle will be totally reflected. Light 45 transmitted into material 40 can be absorbed or further transmitted into and absorbed by an absorbed by an optional second material 50, (e.g. a nonfluorescent dye or pigment, polymer, amorphous or crystalline semiconductor, etc.). After several reflections within the waveguide, traversing the path along material 40, the high-angle light 25 will be substantially eliminated, whereas low angle light 30 will be substantially transmitted, such that material 40 acts as an angle filter.

Light of wavelength 2 remaining in the waveguide may propagate further to the vicinity of the sample 35, where it may evanescently illuminate (e.g., excite fluorescence) in sample components in close proximity to the waveguide, without illuminating sample components further from the waveguide surface 55. Sample fluorescence at wavelength 3 60 is emitted toward the microscope objective 10 where it is collected and analyzed by conventional means.

It is common practice in high-resolution optical microscopy to use immersion objectives, which use a drop of water or oil 65 between the objective lens and the microscope slide or cover glass to increase the numerical aperture of the lens and improve resolution. In this case, a layer of material 70 that is transparent to the sample fluorescence wavelength 3 and of refractive index lower than that of the waveguide 5 can be disposed upon the waveguide surface closest to the microscope objective. The thickness of layer 70 is typically chosen such that the evanescent field of excitation light 30 does not penetrate significantly to the outer surface of the layer, and so does not scatter from the meniscus of the immersion fluid droplet.

It is common in many microscopy applications to observe samples immersed in fluids, such as serum, cell growth media, buffers, and the like. In such cases, a fluid reservoir 75 can be constructed upon the waveguide. To reduce scatter 80 of pump light 30 by the reservoir structure into the reservoir, the portion of the reservoir in contact with the waveguide can be fabricated from a material whose refractive index closely matches that of the contained fluid 85. Alternatively, as shown in FIG. 8, the reservoir structure can be fabricated with a reentrant profile, and of a material absorbent to wavelength 2, such that scattered light is largely intercepted by the reservoir structure and absorbed.

Using the teaching provided herein, numerous modifications will be available to one of skill in the art. For example, the coupler shown as 20 in Figure can be an active or passive coupler. When the coupler is an active structure, it converts primary excitation energy, e.g., light of wavelength 1 directed approximately perpendicular to the waveguide surface, into secondary excitation light at wavelength 2, directed roughly parallel to the waveguide surface. In certain embodiments this can be accomplished by use of a fluorophore. Fluorophores are well known to those of skill in the art and include, but are not limited to organic or inorganic molecules; atoms; ions imbedded in a host; dielectric, semiconductor, or metallic nanoparticles; semiconductor layers, and the like. Illustrative fluorophores include, but are not limited to cyanine dyes, coumarin dyes, fluoresceine and its derivatives, rhodamines (rhodamine and rhodamine derivatives), Texas red dyes, pyrene and pyrene derivatives, and the like.

In general, fluorophores are available that are useful with various excitation light sources and emission wavelengths used in a microscopes are well known. In one example, polyimide materials are available that have effective fluorescence at wavelengths from 473 nm to 850 nm, or from 450 nm to 800 nm, covering essentially the entire visible spectrum. However, fluorophores in the near infrared and ultraviolet may be employed, given suitable circumstances with respect to the sample, and the available sources of illumination and detection.

Other active couplers include, but are not limited to optically pumped lasers, electrically pumped light emitting diodes (LED), diode lasers, and the like which convert an electrical primary excitation source into an optical excitation source. In certain embodiments the coupler may be a passive structure that simply captures and redirects primary excitation light (15), chosen in such cases to be of wavelength 2. Suitable passive couplers include, but are not limited to one or more of the following: microfabricated lenses, prisms, facets, gratings, mirrors, gradient index structures, scattering structures. Alternate locations for these various couplers include either or both planar surfaces of the waveguide, or the edge of the waveguide.

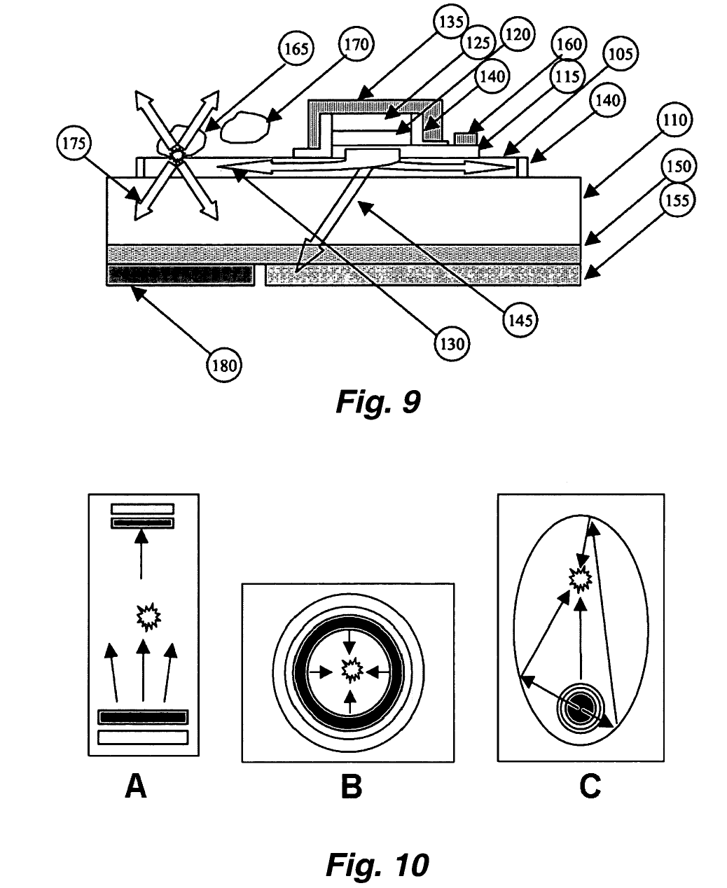

Another illustrative implementation of the invention, using an active electrical-to-optical coupler, is shown schematically in FIG. 9. A planar optical waveguide 105 is fabricated upon a substrate 110 of lower index of refraction. Upon the waveguide is fabricated a light emitting diode, comprising lower contact 115, light emitting 120, and upper contact 125 layers, such that a fraction of the emission from the LED 130 enters the waveguide layer, and is trapped within it by total internal reflection at the upper and lower surface of the waveguide. Light emission from the top and side surfaces of the LED structure can be blocked by contact metal 135 isolated from parts of the structure by dielectric layers 140, to avoid excitation of fluorescent species not in close proximity to the waveguide. Light 145 emitted from the bottom of the LED, that is not captured within the waveguide, can be absorbed at the lower surface of the device by a combination of antireflection 150 and/or absorption 155 layers, to avoid reflection back toward the sample.

A second electrical contact to the device can be made through contact pad 160. In various embodiments the sample is placed directly on the waveguide surface, such that fluorescent species or regions of a sample in close proximity to the surface 165 fluoresce, whereas species or regions of a sample distant from the surface 170 are not excited by the evanescent field and do not fluoresce. The fluorescence 175 can be observed through common microscope optics, from below the waveguide if the waveguide and substrate are transparent to the fluorescent light, or from above the waveguide. Optionally, a spectral filter 180 can be added, to distinguish different fluorescent species, or to further separate fluorescence from background light. In sensing applications, the fluorescence may be detected by photodetectors, image sensors, or visually. As in the implementation shown in FIG. 8, a sample reservoir designed for low scatter can be incorporated into the implementation of FIG. 9. This has been omitted from FIG. 9 for clarity.

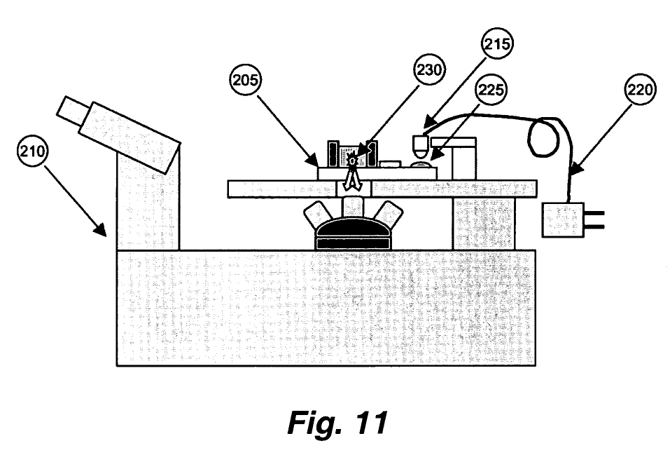

One way to implement the embodiment of FIG. 9 is to fabricate it from alloys of (Al,Ga,In)N, grown epitaxially on a sapphire substrate, similar to LED devices already mass produced for lighting and display purposes. Unlike common LEDs, however, in various embodiments the metal contacts would be completely opaque, and extend over the edges of the LED structure, to eliminate LED emission into the sample area. Light scattering at the lower surface of the substrate would be eliminated with antireflection and/or absorbing layers deposited onto the substrate. Epitaxial layer thicknesses can be optimized for efficient coupling of LED emission into the waveguide and efficient coupling to the sample via the evanescent field. The shape and size of the LED, and the waveguide layer, can be tailored to obtain strong and uniform illumination of the sample area, depending on the intended use. Some possible options are shown in plan view, in FIG. 10, panel A. This panel shows a stripe geometry, capable of illuminating a relatively large area. A second LED structure is shown, operated as a photodiode to monitor the optical excitation power in the waveguide. FIG. 10, panel B shows a ring geometry to provide higher illumination intensity over a smaller area. FIG. 10, panel C shows a disk-shaped LED at one focus of an elliptical mirror, and the sample placed at the other focus, to achieve intense illumination while keeping the sample away from the heat generated by the LED's electrical power dissipation. The elliptical mirror can be formed by etching the waveguide layer to the desired shape, then coating the sidewalls with reflective materials. Similar focusing or collimating designs may be fabricated into the implementation illustrated in FIG. 8 by etching or polishing the waveguide to shape, followed by application of reflective or semi-reflective coatings to the edges.

Another approach to implement the embodiment illustrated in FIG. 9 is to use other material systems for excitation at longer wavelengths than accessible with (Al,GA,In)N, such as (Al,Ga,In)(As,P) on GaAs or GaP substrates. In such cases, the low refractive index material immediately below the waveguide layer can be formed by oxidation of AlAs or AlGaAs to AlOₓ, or by transferring the semiconductor LED structure, with or without a semiconductor waveguide layer, onto a new lower refractive index substrate such as glass or plastic. Such epitaxial transfer or wafer fusion techniques allow wafer scale fabrication of devices to access excitation wavelengths from the deep ultraviolet into the near infrared.

Possible variations include incorporating the LED/waveguide structure into or onto microscope slides, culture dishes, microarray plates, and other common sample handling devices, for easy adaptation to a range of applications.

FIG. 11 shows schematically one version of the invention in use on an inverted optical microscope. The TIRFM device (205) of, e.g., FIG. 8 rests on the stage of an inverted microscope 210. An LED 215 powered by a simple power supply 220 illuminates the coupling structure 225. Fluorescence from cells within a droplet of sample 230 resting on the TIRFM chip is collected by the objective lens of an inverted microscope 210.

It will be appreciated that the TIRFM devices described herein provide a mass-producible component, that can be powered by a simple power supply or battery. The entire device is robust, alignment-free, and inexpensive enough to be expendable. It may be used to add TIRFM capability to standard fluorescence microscopes, and with a simple emission filter included in the device, it may be used to add fluorescence and TIRFM capability to common optical microscopes. Excitation wavelengths from the deep ultraviolet to the near infrared are available, by choice of the materials from which the device is fabricated. The device may be easily incorporated into a wide range of sample cells or microscope slides, and adaptations of the basic invention form the basis for portable, sensitive, and highly multiplexed biochemical sensors.

In certain embodiments, the TIRFM device is contemplated for use in measuring microcantilever deflections. Thus, it will be appreciated that, in certain embodiments, the wave guide can comprise one or more cantilevers in the microcantilever array (e.g., disposed in a reservoir, etc.). After the sample is contacted to the array resulting in specific binding of anti-allergen IgE, the microcantilever array can be contacted with, e.g., a fluorescently labeled antibody that specifically binds to the captured IgE thereby placing a fluorescent species in close proximity to the microcantilever surface where it can be excited by the evanescent field and produce a signal indicating the presence of IgE (or other analyte) on the microcantilever.

In certain embodiments, the microcantilever(s) can be fabricated so that they incorporate a fluorescent material or have such a material affixed. Deflection of the microcantilever (e.g., in response to antigen binding) can be detected/quantified using, for example, the methods and means of detecting sample distance (e.g., from the waveguide) described above.

It will also be noted that while the TIRFM device described herein is contemplated for use in measuring microcantilever deflections, the device need not be limited to such use and will generally be of useful to provide improved sensitivity and contrast in fluorescence microscopy, and to allow examination of restricted cross sections of fluorescence microscopy samples. Thus, the invention is also useful for biochemical sensing (e.g. detecting antibody binding, nucleic acid hybridization, ligand/receptor binding, etc.), with applications in drug development, clinical screening, environmental monitoring, forensics, security, and the like.

It will also be appreciated that the invention is not limited to the specifically illustrated embodiments. Using the teachings provided herein, other embodiments will be available to one of skill in the art.

Fabrication

Fabrication of TIRFM apparatus.

The TIRFM apparatus described herein can be fabricated using standard methods for optical coating and/or assembly and/or microfabrication. Such methods include, but a not limited to lamination, cementing, and welding methods as well as photolithographic etching and/or deposition methods, e.g. as described below.

Fabrication of Microcantilever Arrays.

In one preferred embodiment, the microcantilever array(s) are fabricated using micromachining processes (e.g. photolithography) well known in the solid state electronics industry. Commonly, microdevices are constructed from semiconductor material substrates such as crystalline silicon, widely available in the form of a semiconductor wafer used to produce integrated circuits, or from glass. Because of the commonality of material(s), fabrication of microdevices from a semiconductor wafer substrate can take advantage of the extensive experience in both surface and bulk etching techniques developed by the semiconductor processing industry for integrated circuit (IC) production.

Surface etching, used in IC production for defining thin surface patterns in a semiconductor wafer, can be modified to allow for sacrificial undercut etching of thin layers of semiconductor materials to create movable elements. Bulk etching, typically used in IC production when deep trenches are formed in a wafer using anisotropic etch processes, can be used to precisely machine edges or trenches in microdevices. Both surface and bulk etching of wafers can proceed with “wet processing”, using chemicals such as potassium hydroxide in solution to remove non-masked material from a wafer. For microdevice construction, it is even possible to employ anisotropic wet processing techniques that rely on differential crystallographic orientations of materials, or the use of electrochemical etch stops, to define various channel elements.

Another etch processing technique that allows great microdevice design freedom is commonly known as “dry etch processing”. This processing technique is particularly suitable for anistropic etching of fine structures. Dry etch processing encompasses many gas or plasma phase etching techniques ranging from highly anisotropic sputtering processes that bombard a wafer with high energy atoms or ions to displace wafer atoms into vapor phase (e.g. ion beam milling), to somewhat isotropic low energy plasma techniques that direct a plasma stream containing chemically reactive ions against a wafer to induce formation of volatile reaction products.

Intermediate between high energy sputtering techniques and low energy plasma techniques is a particularly useful dry etch process known as reactive ion etching. Reactive ion etching involves directing an ion containing plasma stream against a semiconductor, or other, wafer for simultaneous sputtering and plasma etching. Reactive ion etching retains some of the advantages of anisotropy associated with sputtering, while still providing reactive plasma ions for formation of vapor phase reaction products in response to contacting the reactive plasma ions with the wafer. In practice, the rate of wafer material removal is greatly enhanced relative to either sputtering techniques or low energy plasma techniques taken alone. Reactive ion etching therefore has the potential to be a superior etching process for construction of microdevices, with relatively high anistropic etching rates being sustainable. The micromachining techniques described above, as well as many others, are well known to those of skill in the art (see, e.g., Choudhury (1997) The Handbook of Microlithography, Micromachining, and Microfabrication, Soc. Photo-Optical Instru. Engineer, Bard & Faulkner (1997) Fundamentals of Microfabrication). In addition, examples of the use of micromachining techniques on silicon or borosilicate glass chips can be found in U.S. Pat. 5,194,133, 5,132,012, 4,908,112, and 4,891,120.

In one embodiment, the channel is micromachined in a silicon wafer using standard photolithography techniques to pattern the cantilever, chambers, optional channels, sample processing chambers, connection ports, and the like. In certain embodiments ethylene-diamine, pyrocatechol (EDP) can be used for a two-step etch and a Pyrex 7740 coverplate can be anodically bonded to the face of the silicon to provide a closed liquid system. In this instance, liquid connections can be made on the backside of the silicon.

As indicated above, in certain embodiments, the device is fabricated from glass, quartz, or other similar material.

Attachment of Antigen or Other Binding Moieties.

Many methods for immobilizing biomolecules (e.g., antigens, antibodies, etc.) to a variety of solid surfaces are known in the art. The desired component can be covalently bound, or noncovalently attached through specific or nonspecific bonding.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Ichiro Chibata (1978) Immobilized Enzymes, Halsted Press, New York, and Cuatrecasas, (1970) J. Biol. Chem. 245: 3059.

In addition to covalent bonding, various methods for noncovalently binding a component (e.g. an antigen) can be used. Noncovalent binding is typically nonspecific absorption of a compound to the surface. In various embodiments the cantilever surface is blocked with a second compound to prevent nonspecific binding of target. . Alternatively, the surface is designed such that it nonspecifically binds one component but does not significantly bind another. For example, a surface bearing a lectin such as concanavalin A will bind a carbohydrate containing compound but not a labeled protein that lacks glycosylation. Various solid surfaces for use in noncovalent attachment of assay components are reviewed in U.S. Pat. Nos. 4,447,576 and 4,254,082.

In certain embodiments, the binding moiety (e.g., antigen, anti-IgE antibody, etc.) is immobilized on the cantilever(s) by the use of a linker (e.g. a homo- or heterobifunctional linker). Linkers suitable for joining biological binding partners are well known to those of skill in the art. For example, a protein or nucleic acid molecule may be linked by any of a variety of linkers including, but not limited to a peptide linker, a straight or branched chain carbon chain linker, or by a heterocyclic carbon linker. Heterobifunctional cross linking reagents such as active esters of N-ethylmaleimide have been widely used (see, for example, Lerner et al. (1981) Proc. Nat. Acad. Sci. USA, 78: 3403-3407 and Kitagawa et al. (1976) J. Biochem., 79: 233-236, and Birch and Lennox (1995) Chapter 4 in Monoclonal Antibodies: Principles and Applications, Wiley-Liss, N.Y.).

In one embodiment, the antigen, binding moiety, or antibody is immobilized on the cantilever utilizing a biotin/avidin interaction. In one approach, biotin or avidin with a photolabile protecting group can be attached to the cantilever surface. Irradiation of the distinct cantilevers results in coupling of the biotin or avidin to the illuminated cantilever(s) at that location. Then, the antigen or other binding moiety, bearing a respective biotin or avidin is placed into the channel whereby it couples to the respective binding partner and is localized on the irradiated cantilever. The process can be repeated at each distinct location it is desired to attach a binding partner.

Another suitable photochemical binding approach is described by Sigrist et al. (1992) Bio/Technology, 10: 1026-1028. In this approach, interaction of ligands with organic or inorganic surfaces is mediated by photoactivatable polymers with carbene generating trifluoromethyl-aryl-diazirines that serve as linker molecules. Light activation of aryl-diazirino functions at 350 nm yields highly reactive carbenes and covalent coupling is achieved by simultaneous carbene insertion into both the ligand and the inert surface. Thus, reactive functional groups are not required on either the ligand or supporting material.

In still another approach, the microcantilever(s) are coated with a thin layer of epoxy (Epotek 350) in order to cover the cantilever surface with an organic coating. A protocol for coating the such surfaces with the epoxy is described by Liu et al. (1996) J. Chromatogr. 723: 157-167. The coated microcantilever(s) can then be flushed with a specific binding moiety solution. The solution is allowed to react with the microcantilever(s) to bind the allergen or other binding moiety via hydrophobic and electrostatic interactions.

Blocking Protein Attachment.

In certain embodiments the microcantilever arrays comprise negative control microcantilevers that are treated to prevent attachment of protein. Methods of treating surfaces to prevent protein attachment are known to those of skill in the art. Such methods include, but are not limited to coating the surface with materials such as pp4G, plasma-polymerized tetraglyme (see, e.g., Hanein et al. (2001) Sensors and Actuators B 81: 49-54), surfactants, and the like.

Kits

In certain embodiments, this invention provides kits for practicing the various methods described herein. The kits can include, for example, the microcantilever array, and/or a TIRFM device as described herein. In various embodiments the microcantilever may be provided as a component of a TIRFM device (e.g., disposed in a well on a waveguide as described herein).

Where the microcantilever device and/or TIRFM device incorporates reservoirs, the reservoirs can, optionally, contain one or more buffers, labels, and/or bioactive agents (e.g., anti-IgE antibody, fluorophore, etc.) as required. In certain embodiments the bioactive agent or other agent is provided in a dry rather than a fluid form so as to increase shelf life.

The kits can optionally further comprise buffers, syringes, sample collectors and/or other reagents and/or devices to perform one or more of the assays described herein.

The components comprising the kits are typically provided in one or more containers. In certain preferred embodiments, the containers are sterile, or capable of being sterilized (e.g. tolerant of on site sterilization protocols).

The kits can be provided with instructional materials teaching users how to use the device of the kit. For example, the instructional materials can provide directions on utilizing the assay device (e.g. microcantilever array, and/or array reader) to diagnose one or more allergies in a subject (e.g., a human patient) and/or for the operation of a TIRFM device.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A device for detecting and characterizing an allergy, said device comprising:
a sample chamber; and
an array of microcantilevers wherein microcantilevers comprising said array have affixed thereto antigen such that there is a different species of antigen for each allergy it is desired to detect, and different species of antigen are on different microcantilevers in said array, wherein the free ends of the microcantilevers project into the sample chamber.

2. The device of claim 1, wherein said device comprises at least 4 microcantilevers each having affixed thereto different binding moieties.

3. The device of claim 1, wherein said device comprises at least 10 microcantilevers each having affixed thereto different binding moieties.

4. The device of claim 1, wherein said device comprises at least 100 microcantilevers each having affixed thereto different binding moieties.

5. The device of claim 1, wherein said device comprises negative control microcantilevers treated to resist binding by protein.

6. The device of claim 1 or 5, wherein said device comprises positive control microcantilevers having attached thereto an antibody that binds IgE antibodies.

7. The device of claim 6, wherein the antibody that binds to IgE antibodies is a single chain antibody.

8. The device of claim 6, wherein the antibody that binds to IgE antibodies is a monoclonal antibody.

9. The device of claim 1, further comprising a first means of detecting deflection of a cantilever when binding moieties on the cantilever bind a target analyte.

10. The device of claim 9, further comprising a second means of detecting deflection of a cantilever when binding moieties on the cantilever bind a target analyte.

11. The device of claim 9 or 10, wherein said first means and said second means are independently selected from the group consisting of an optical detection means, a piezoresistive detection means, a piezoelectric detection means, and an evanescent wave detection means.

12. The device of claim 1, wherein said allergen is selected from the group consisting of a pet allergen, dust, mold spores, pollen, a food allergen, and an insect bite allergen.

13. A method of identifying an allergy in a subject, said method comprising:
providing a biological sample from said subject comprising IgE antibodies;
contacting said biological sample or a component thereof with a device according to any one of claims 1 through 12; and
detecting deflection of one or more cantilevers in the microcantilever array in response to binding by IgE where binding of the cantilever indicates that said subject has an allergic response to the antigen present on the deflected cantilever.

14. The method of claim 13, wherein said detecting comprises a method selected from the group consisting of detecting an optical signal, detecting a piezoresistive signal, detecting an optical signal, detecting an evanescent wave signal.

15. The method of claim 14, wherein said detecting comprises utilizing at least two different detection methods.

16. The method of claim 13, wherein said sample comprises whole blood, plasma, or serum.

17. A device for detecting the presence, absence, or quanity of a plurality of analytes, said device comprising:
a sample chamber; and
an array of microcantilevers wherein micocantilevers comprising said array have affixed thereto binding moieties such that there is a different species of binding moiety that specifically or preferentially binds each species of analyte that is to be detected; and different species of binding moiety are on different microcantilevers in said array, wherein the free ends of the microcantilevers project into the sample chamber.

18. The device of claim 17, wherein said device comprises at least 4 microcantilevers each having affixed thereto different binding moieties.

19. The device of claim 17, wherein said device comprises at least 10 microcantilevers each having affixed thereto different binding moieties.

20. The device of claim 17, further comprising a first means of detecting deflection of a cantilever when binding moieties on the cantilever bind a target analyte.

21. The device of claim 20, further comprising a second means of detecting deflection of a cantilever when binding moieties on the cantilever bind a target analyte.

22. The device of claim 20 or 21, wherein said first means and said second means are independently selected from the group consisting of an piezoresistive detection means, a piezoelectric detection means, and an optical detection means.

23. The device of claim 22, wherein said first means and said second means are optical detection means selected from the group consisting of means to detect optical beam deflection, means to detect optical phase shift, means to detect optical intensity shift, and means to detect evanescent field excitation of fluorescence.

24. A device for supporting a sample and for providing evanescent field excitation of fluorescence in total internal reflectance microscopy (TIRFM), said device comprising:
a substantially planar optical waveguide comprising two substantially parallel surfaces; and
an active optical coupler affixed or juxtaposed to said waveguide such that light generated from said coupler enters said waveguide, where said active optical coupler is not a fluorophore.

25. A device for supporting a sample and for providing evanescent field excitation of fluorescence in total internal reflectance microscopy (TIRFM), said device comprising:
a substantially planar optical waveguide comprising two substantially parallel surfaces;
an active optical coupler affixed or juxtaposed to said waveguide such that light generated from said coupler enters said waveguide; and
an angle filter comprising a material whose refractive index is between that of the waveguide and air, where said angle filter is disposed on a surface of said waveguide to substantially reduce light propagating in the waveguide within a predetermined range of angles.

26. A device for supporting a sample and for providing evanescent field excitation of fluorescence in total internal reflectance microscopy (TIRFM), said device comprising:
a substantially planar optical waveguide comprising two substantially parallel surfaces; and
a passive optical coupler affixed or juxtaposed to said waveguide such that light provided from said coupler enters said waveguide.

27. The device of any one of claims 24 or 25, wherein said active optical coupler is an electrically driven coupler or an optically pumped laser.

28. The device of claim 27, wherein said active optical coupler is an electrically driven coupler selected from the group consisting of a light emitting diode (LED), a laser diode, an electroluminescent device, and a microplasma discharge device.

29. The device of claim 25, wherein said active optical coupler is a fluorophore.

30. The device of claim 26, wherein said passive optical coupler is selected from the group consisting of a lens, a prism, a facet, a grating, a mirror, a gradient index structure, and a scattering structure.

31. The device of any of claims 24 or 26, wherein said device further comprises an angle filter comprising a material whose refractive index is between that of the waveguide and air, where said angle filter is disposed on a surface of said waveguide to substantially reduce light propagating in the waveguide.

32. The device of claim 31, wherein said angle filter substantially eliminates or reduces light propagating in the waveguide at an angle below a critical angle, measured relative to a line perpendicular to the waveguide surface and drawn into the waveguide, said critical angle ranging from about 35 degrees to about 70 degrees.

33. The device of claim 25, wherein said angle filter substantially eliminates or reduces light propagating in the waveguide at an angle below a critical angle, measured relative to a line perpendicular to the waveguide surface and drawn into the waveguide, said critical angle ranging from about 35 degrees to about 70 degrees.

34. The device of any one of claims 24, 25, or 26, wherein said waveguide has an index of refraction of about 1.4 or more.

35. The device of any one of claims 24, 25, or 26, wherein said waveguide ranges in thickness from about 50 μm to about 1 mm.

36. The device of claim 35, wherein said waveguide ranges in thickness from about 50 μm to about 500 μm.

37. The device of claim 35, wherein said waveguide ranges in thickness from about 100 μm to about 200 μm.

38. The device of any one of claims 24, 25, or 26, wherein said waveguide comprises a material selected from the group consisting of glass, plastic, and a crystalline material.

39. The device of claim 38, wherein said waveguide comprises a crystalline material selected from the group consisting of quartz, sapphire, silicon carbide, calcium fluoride, aluminum nitride, gallium nitride, aluminum gallium nitride, magnesium fluoride, and lithium niobate.

40. The device of claim 38, wherein said waveguide comprises a coverslip.

41. The device of any one of claims 24, 25, or 26, wherein said device further comprises a substantially planar low refractive index material immediately below the waveguide.

42. The device of claim 41, wherein said low refractive index material has a refractive at least 0.05 below that of the waveguide, and a thickness of at least 1 μm.

43. The device of claim 24, 25, or 26, wherein said optical coupler is laminated to said waveguide.

44. The device of any one of claims 24, 25, or 26, wherein said device further comprises a means for supporting or affixing a sample such that all or a portion of said sample is exposed to an evanescent field from said optical waveguide.

45. The device of claim 44, wherein said means comprises one or more fluid reservoirs.

46. The device of any one of claims 24, 25, or 26, wherein said device further comprises a means to measure intensity of an excitation light.

47. The device of claim 46, wherein said means to measure excitation intensity comprises one or more fluorophores that are excited by the same evanescent field used to excite the sample of interest, and that emit fluorescence that is proportional to excitation intensity.

48. The device of claim 47, wherein said fluorophores are distributed on the waveguide surface in known and easily distinguishable patterns.

49. The device of claim 46, wherein said means to measure excitation intensity comprises a photodiode that intercepts a portion of the excitation light.

50. The device of any one of claims 24, 25, or 26, wherein said device further comprises a means to quantify sample distance from the waveguide surface.

51. The device of claim 50, wherein said means to quantify sample distance comprises fluorescent markers at known distances from the waveguide surface.

52. The device of claim 50, wherein said means to quantify sample distance comprises two or more couplers emitting light at significantly different wavelengths, in conjunction with a sample fluorophore that can be excited by light at significantly different wavelengths.

53. The device of any one of claims 24, 25, or 26, wherein said device further comprises structures that reduce scattering of excitation light at boundaries of fluids disposed on the waveguide surface, or at boundaries of structures that contain those fluids.

54. The device of claim 53, wherein said structures comprise an antireflection layer and/or an absorption layer.

55. The device of claim 53, wherein said structures are selected from the group consisting of structures fabricated from material with an index of refraction approximately equal to that of the contained fluid, and structures with reentrant profiles such that light scattered at the point of contact between the structure and the substrate is subsequently intercepted and absorbed by another part of the structure.

56. The device of any one of claims 24, 25, or 26, wherein the planar surface opposite the sample is coated with a smooth and transparent layer of thickness greater than approximately one micrometer and index of refraction lower than that of the waveguide, such that light trapped by total internal reflection in the waveguide does not penetrate evanescently to the surface of said layer.

57. The device of any one of claims 24, 25, or 26, wherein a solid or liquid layer is disposed on the substrate such that excitation light propagating within the waveguide within some range of propagation angles relative to the planar surface is transmitted out of the substrate and into the solid or liquid layer, and is subsequently transmitted away from the device or absorbed.

58. The device of any one of claims 24, 25, or 26, wherein a planar surface opposite the sample is coated with an absorptive or reflective optical filter, such that only sample fluorescence of selected wavelengths is transmitted through the filter.

59. The device of any one of claims 24, 25, or 26, wherein said device is disposable.
